SEQUENCE LISTING (5 sequences)

SEQ ID NO: 1 - protein, 7 aa, Homo sapiens
PYGNHSY

SEQ ID NO: 2 - protein, 9 aa, Homo sapiens
FGTINNFRL

SEQ ID NO: 3 - protein, 12 aa, Homo sapiens
LYCSGGLRFFWD

SEQ ID NO: 4 - protein, 9 aa, Homo sapiens
QTVLLLHAL

SEQ ID NO: 5 - protein, 13 aa, Homo sapiens
RLVPYGNHSYLES

(19) United States
(12) Patent Application Publication
(10) Pub. No.: US 2023/0321188 A1
(43) Pub. Date: Oct. 12, 2023

(54) AUTOPHAGY INDUCING PEPTIDES

(71) Applicant: TRANSLATIONAL HEALTH SCIENCE AND TECHNOLOGY INSTITUTE, Faridabad (IN)

(72) Inventors: Shailendra ASTHANA, Faridabad (IN); Charu SURI, Faridabad (IN); Manjula KALIA, Faridabad (IN); Amit AWASTHI, Faridabad (IN); Ajit CHANDE, Bhauri (IN); Ambadas RODE, Faridabad (IN); Surendra kumar PRAJAPAT, Faridabad (IN); Srikanth SADHU, Faridabad (IN); Rajdeep DALAL, Faridabad (IN); Vipin BHARDWAJ, Bhauri (IN); Divya OJHA, Bhauri (IN)

(21) Appl. No.: 18/022,164

(22) PCT Filed: Aug. 19, 2021
(86) PCT No.: PCT/IN2021/050799

§ 371 Date: Feb. 19, 2023

(30) Foreign Application Priority Data
Aug. 19, 2020 (IN) 202011007117

(51) Int. Cl. A61K 38/17; A61P 31/12; A61P 1/12; A61P 31/04

(52) U.S. Cl. CPC A61K 38/177; A61P 31/12; A61P 1/12; A61P 31/04

(57) ABSTRACT

The present invention discloses a novel engineered peptides P3T, P7T, P8T, P9T, P13T and P8_d1 of Seq ID No. 1 to combinations comprising one or more said peptides and compositions comprising one or more said peptides with pharmaceutically acceptable carriers and excipients and their use. The peptides and composition comprising said peptides induce autophagy by binding the protein GAPR-1 (−ve regulator of autophagy). Endogenously, GAPR-1 acts by tethering critical autophagy protein Beclin-1 (+ve regulator of autophagy) at the golgi and arrest autophagy. The peptide reported here binds GAPR-1 to free Beclin-1 and subsequently induce autophagy. The peptide can be used to induce or enhance autophagy and finds therapeutic application in a broad spectrum of disease pathologies that benefit from augmented autophagy, including obesity, diabetes, NASH, cancer, cardiomyopathy, neurodegenerative diseases, IBD and pathogenic infections.

### FIELD OF THE INVENTION

The present invention relates generally to the field of biotechnology. In, the present invention relates to novel peptides, combination of the novel peptides, a composition comprising the peptide(s) and a process to manufacture the same. The novel peptide and its composition can be used for induction of autophagy in a m-Tor independent manner.

### BACKGROUND OF THE INVENTION

Autophagy is a critical catabolic process which is necessary for maintaining cellular homeostasis. Its dysregulation has been implicated in various infectious and metabolic diseases. Beclin-1 is an essential autophagy protein which forms two essential complexes with phospho-inositol-III called PI3K complex-I and PI3K complex-II. These complexes are required for the initiation of autophagosome formation and fusion of autophagosome with the lysosome, respectively. GAPR-1 has been reported as an endogenous inhibitor of autophagy. It acts by tethering Beclin-1, an important autophagy protein to golgi complex, hence making it unavailable to initiate autophagy. It has been previously reported that autophagy can be induced by disrupting the interaction between beclin-1 and Gapr-1. Breaking the complex frees beclin-1 form golgi, allowing it to form both PI3K complex-1 and PI3k complex-II.

However, there is a need for compounds or peptides that can utilise this pathway for induction of autophagy and thereby help in intervention of various diseases where autophagy is indicated.

### OBJECT OF THE INVENTION

An object of the invention is to provide novel engineered peptide for autophagy induction and combinations thereof and compositions comprising one or more said peptides with pharmaceutically acceptable carriers and excipients.

Yet another object is to induce autophagy by the novel engineered peptides of the present invention.

### SUMMARY OF THE INVENTION

Autophagy is a critical catabolic process which is necessary for maintaining cellular homeostasis. Its dysregulation has been implicated in various infectious and metabolic diseases. The invention of the present application provides novel engineered peptides of Seq Id. No. 1 to 5 selected and combinations thereof; and compositions comprising one or more said peptides with pharmaceutically acceptable carriers and excipients that can be used to induce autophagy in diseased states.

### BRIEF DESCRIPTION OF THE FIGURES

FIG. 1 depicts modelling of protein-protein interaction mode of GAPR-1 and Beclin-1 and Predicted Binding modes of Gapr-1 with Beclin-1 ECD.

FIG. 2 depicts prediction of Hot-spot amino acids.

FIGS. 3A-E depicts MicroScaleThermophoresis (MST) of peptides P3T, P7T, P8T, P9T and P13T binding with Gapr-1. Peptide P3T was found to bind to purified Gapr-1 with the Kd of 0.21 μM (3A); P7T with Kd of 0.13 μM (3B), P8T with Kd of 0.12 μM (3C); P9T with Kd of 1.56 μM(3D); and P13T with Kd of 0.07 μM (3E) FIGS. 4A-D depicts the Autophagy induction by PT series of peptides (P3T, P7T, P8T, P9T and P13T): Huh7 (A-B)/Hela (C-D) cells were treated with peptides at 10.0 μM concentration and Torin (1 μM) for 6 h, washed in PBS and analyzed through SDS-PAGE and western blot using LC3 and GAPDH antibodies. LC3-I and LC3-II, Microtubule-associated protein 1A/1B -light chain 3, GAPDH (internal control).

FIG. 5 depicts mTOR independent activation of autophagy Huh7 cells were treated with peptides (10 μM) and torin1 (1 μM) for 6 h and lysed in lysis buffer (1mM EDTA, 20 mM Tris-HCl pH 7.4, 250 mM NaCl, PMSF, and PI). Western blot was performed by GAPDH (internal control), P-T386S6K1 and S6K1 antibodies. The peptides were tested for inhibition of mTOR by testing for phosphorylation of enzyme p70S6K. The phosphorylation of p-p70S6K at threonine 389 has been used as a hallmark of activation by mTOR. Peptides P7T, P9T and P13T did not interfere with the phosphorylation of p70S6K and mTOR activity. Hence, these peptides induce autophagy in an mTOR dependent manner.

FIG. 6 depicts percentage inhibition of Salmonella in presence of peptides. FIG. 7 depicts In vivo effect of P7T on S. typhimurium infection and disease progression in FVB mice.

FIG. 8 depicts In vivo effect of P7T on mice model of IBD. A. Representative figure of body weight changes in peptide P7T (20 mg/Kg) treated mice verses vehicle treated in experimental colitis induced by 2% DSS given in drinking water for 7 days. B. Colon length reduction in mice treated with P7T and vehicle. C-D. The rectal bleeding and diarrhea score of mice treated with P7T and vehicle.

FIG. 9 depicts comparison of peptides of the present invention with known peptides for autophagy induction.

FIG. 10 depicts the anti-HIV activity of the present invention in virus production and infectivity assay

### DETAILED DESCRIPTION OF THE INVENTION

The instant application contains a Sequence Listing which has been submitted in PATENTIN and is hereby incorporated by reference in its entirety. Said PATENTIN version, created on Aug. 19, 2021, is named Sequences listing .txt and is 3 KB in size.

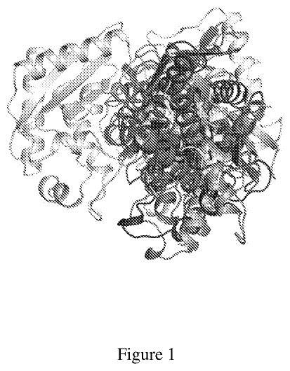

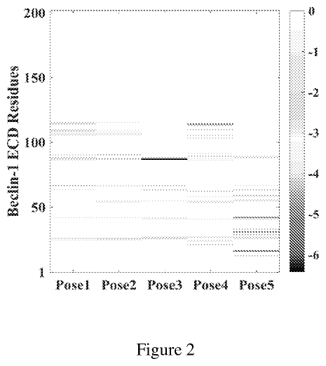

The present invention is accompanied by figures. FIG. 1 depicts modelling of protein-protein interaction mode of GAPR-1 and Beclin-1 and Predicted Binding modes of Gapr-1 with Beclin-1 ECD. The present invention also depicts at FIG. 2 the prediction of Hot-spot amino acids by computational aided methods. Based on such computational methods, the present invention arrived at novel peptides, that were engineered and subsequently synthesised and surprisingly, found to have superior autophagy induction properties.

The present invention discloses novel engineered peptide for autophagy induction, said peptide being selected from group comprising P3T, P7T, P8T, P9T, P13T and P8_d1 of Seq ID No. 1 to 6 or their combinations.

The present invention is drawn to a novel engineered peptide comprising beclin-1 residues 332-338 and a heterologous moiety to promote therapeutic stability or delivery of the compound to induce autophagy. The novel peptides were engineered after identifying high affinity peptide through extensive computational methods. These peptides were further validated through standard in vitro methods and in vivo methods for their ability to induce autophagy. The peptide of the present invention also inhibits viral replication using HeLa cells infected with two alphaviruses, Sindbis virus and chikungunya virus, and one flavivirus, West Nile virus and other viruses including Coronavirus.

In an aspect the present invention of the present application discloses novel engineered peptide for autophagy induction selected from group comprising P3T, P7T, P8T, P9T, P13T and P8_d1 of Seq ID NOS. 1 to 6 and combinations thereof.

The present invention also discloses novel engineered peptide for autophagy induction selected from group comprising P3T, P7T, P8T, P9T and P13T comprising Beclin 1 residues 332-338(PYGNHSY), 282-291 (FGTINNFRL), 351-362(LYCSGGLRFFWD), 310-317(QTVLLLHAL) and 329-341(RLVPYGNHSYLES) respectively immediately flanked on each terminus by no more than 12 (or 6, 3, 2, 1 or 0) naturally-flanking Beclin 1 residues, wherein up to six (or 3, 2, 1 or 0) of said residues 332-338 (PYGNHSY), 282-291 (FGTINNFRL), 351-362(LYCSGGLRFFWD), 310-317(QTVLLLHAL) and 329-341(RLVPYGNHSYLES), respectively may be substituted.

As exemplified herein, peptide and compound activity are tolerant to a variety of additional moieties, flanking residues, and substitutions within the defined boundaries. For example, in some embodiments the peptide is N-terminally flanked with T-N and C-terminally flanked by T. The present invention discloses novel engineered peptide for autophagy induction selected from group comprising P3T, P7T, P8T, P9T and P13T comprising Beclin 1 residues 332-338(PYGNHSY), 282-291 (FGTINNFRL), 351-362(LYCSGGLRFFWD), 310-317(QTVLLLHAL) and 329-341(RLVPYGNHSYLES), respectively.

The present invention discloses a novel engineered peptide for autophagy wherein the peptide is P7T, of Seq ID No. 2.

The present invention also discloses that the combination of the peptides of the present invention has enhanced therapeutic efficacy. The peptide for autophagy induction of the present invention may be in the form a combination of two or more peptides in any manner selected from group comprising P3T, P7T, P8T, P9T, P13T and P8_d1 of Seq ID No. 1 to 6, preferably the combination is of P7T and P13T or a combination of P3T and P13T. From FIG. 4D of the present invention it can be seen that combination of the peptides P3T and P7T (combo 1) and P3T and P13T (combo 2) show enhanced therapeutic efficacy. It was surprisingly found that the combination of the present invention has enhanced activity beyond the aggregation of the activities of the individual peptides. Therefore, the peptides of the present invention may be administered individually or as their combinations.

The present invention envisages within its scope modifications such as backbone modification and replacement, side-chain modifications, and N- and C-terminal modifications, all conventional in the art of peptide chemistry and where chemical modifications of the peptide bonds may be used to provide increased metabolic stability against enzyme-mediated hydrolysis. The present invention envisages within its scope, peptide bond replacements (peptide surrogates), such as trifluoroethylamines, can provide metabolically more stable and biologically active peptidomimetics. The novel engineered peptides P3T, P7T, P8T, P9T and P13T can be modified to constrain the peptide backbone including, for example, cyclic peptides/peptidomimetics, inverse peptides, retro-inverse peptides, stapled peptides, which can exhibit enhanced metabolic stability against exopeptidases due to protected C- and N-terminal ends. Suitable techniques for cyclization include Cys-Cysdisulfide bridges, peptide macrolactam, peptide thioether, parallel and antiparallel cyclic dimers, etc.

Other suitable modifications include acetylation, acylation (e.g., lipopeptides), formylation, amidation, phosphorylation (on Ser, Thr and/or Tyr), etc. which can be used to improve peptide bioavailability and/or activity, glycosylation, sulfonation, incorporation of chelators (e.g., DOTA, DPTA), etc are also envisaged within the scope of the present invention. The present peptides may be PEGylated. PEGylation can be used to increase peptide solubility, bioavailability, in vivo stability and/or decrease immunogenicity. The PEGs that may be used includes a variety of different PEGs: HiPEG, branched and forked PEGs, releasable PEGs; heterobifunctional PEG (with endgroup N-Hydroxysuccinimide (NHS) esters, maleimide, vinyl sulfone, pyridyldisulfide, amines, and carboxylic acids), etc., and such PEGs may have suitable terminal modifications include N-terminal acetyl, formyl, myristoyl, palmitoyl, carboxyl and 2-furosyl, and C-terminal hydroxyl, amide, ester and thioester groups/PEGylation can make the peptide more closely mimic the charge state in the native protein, and/or make it more stable to degradation from exopeptidases and where the novel engineered peptide may also contain atypical or unnatural amino acids, including D-amino acids, L-P-homo amino acids, - β-homo amino acids, N-methylated amino acids, etc.

The peptides of the present invention comprise a first moiety, heterologous to (not naturally flanking) the Beclin 1 peptide. It is hypothesised that the heterologous peptide promotes therapeutic stability or delivery, and optionally, a same or different second moiety, preferably also heterologous to the Beclin 1 peptide. The peptide of the present invention may be N-terminally joined to the first moiety, and C-terminally joined to the second moiety; and where a wide variety of such moieties may be employed, such as affinity tags, transduction domains, homing or targeting moieties, labels, or other functional groups, so as to improve bioavailability and/or activity, and/or provide additional properties; and where the novel engineered peptides that can be in composition with another class are homing biomolecules, such as RGD-4C, CREKA, LyP-1, F3, SMS, IF7, and H2009.1, particularly cancer cell homing or targeting biomolecules, wherein suitable examples are known in the art, e.g. Homing peptides as targeted delivery vehicles.

The present invention also discloses peptides that are derivatised from the peptides of the present invention. As an illustration of P8_d1 is obtained from peptide PBT. Peptide. P8_d1 had 4 amino acid flanking N-terminal and 2 residues on C-Terminal with final sequence KELPLYCSGGLRFFWDNK as provided in Seq Id. No. 6.

The present invention discloses the peptide is P8_d1, of Seq ID No 6.

In an aspect, the novel engineered peptides of the present invention may be obtained by a synthetic process or by recombinant techniques. The peptides of the present invention may be obtained by solid-phase peptide synthesis (SPPS), preferably by protection with Fmoc chemistry followed by deprotection of the Fmoc group at N-terminal first, and if required, deprotection of the side chain protection group. After complete deprotection, the peptide may be cleaved off from the Resin. The synthesised peptide may be purified chromatographic techniques. The present invention also envisages with it scope, the synthesis of peptides of the present invention through recombinant microbial techniques.

In another aspect, the present invention provides a composition comprising one or more said autophagy inducing peptides selected from the group comprising P3T, P7T, P8T, P9T, P13T and P8_d1 of Seq ID No. 1 to 6. with pharmaceutically acceptable carriers and excipients.

Where composition comprises:

(a) one or more novel engineered peptide from the group of autophagy-inducing peptides P3T, P7T, P8T, P9T and P13T comprising Beclin 1 residues 332-338(PYGNHSY), 282-291 (FGTINNFRL), 351-362(LYCSGGLRFFWD), 310-317(QTVLLLHAL) and 329-341(RLVPYGNHSYLES), respectively immediately flanked on each terminus by no more than 12 (or 6, 3, 2, 1 or 0) naturally-flanking Beclin 1 residues, wherein up to six (or 3, 2, 1 or 0) of said residues 332-338, 282-291 (FGTINNFRL), 351-362(LYCSGGLRFFWD), 310-317(QTVLLLHAL) and 329-341(RLVPYGNHSYLES) respectively may be substituted, and a first heterologous moiety,

(b) pharmaceutically acceptable excipients

The composition of the present invention when it is administered orally, parenterally, or topically.

Administration of the compounds of this disclosure or their pharmaceutically acceptable salts, in pure form or in an appropriate pharmaceutical composition, can be carried out via any of the accepted modes of administration or agents for serving similar utilities. Thus, administration can be, for example, orally, nasally, parenterally (intravenous, intramuscular, or subcutaneous), topically, transdermally, intravaginally, intravesically, intracistemally, or rectally, in the form of solid, semi-solid, lyophilized powder, or liquid dosage forms, such as for example, tablets, suppositories, pills, soft elastic and hard gelatin capsules, powders, solutions, suspensions, or aerosols, or the like, preferably in unit dosage forms suitable for simple administration of precise dosages. Solid dosage forms for oral administration include capsules, tablets, pills, powders, and granules. Solid dosage forms, as described above, can be prepared with coatings and shells, such as enteric coatings. Liquid dosage forms for oral administration include pharmaceutically acceptable emulsions, solutions, suspensions, syrups, and elixirs. Compositions for rectal administrations are, for example, suppositories that can be prepared by mixing the compounds of this disclosure with, for example, suitable non-irritating excipients or carriers. They are also parenteral and administered as sterile powders for reconstitution into sterile injectable solutions or dispersions. Dosage forms for topical administration of a compound of this disclosure include ointments, powders, sprays, and inhalants. Ophthalmic formulations, eye ointments, powders, and solutions are also contemplated for the compounds in this disclosure. Compressed gases can be used to disperse a compound of this disclosure in aerosol form.

In an embodiment, the peptides, or combinations thereof as or composition comprising the peptides may be administered in an amount ranging from 1 to 100 mg/Kg to a mammal, preferably in an amount ranging from 1 to 50 mg/Kg to a mammal, in an amount ranging from 1 to 25 mg/Kg to a mammal.

Yet another aspect of the present invention discloses the use of the peptides as disclosed in the application or the composition containing the peptide for induction of autophagy. The present application discloses the use of the novel engineered autophagy-inducing peptides selected from group comprising P3T, P7T, P8T, P9T, P13T and P8_d1 of Seq ID No. 1 to 6; combinations thereof and composition comprising peptides selected from the group of peptides P3T, P7T, P8T, P9T, P13T and P8_d1 of Seq ID No. 1 to 6 pharmaceutically acceptable carriers and excipients for induction of autophagy. The novel engineered peptides of the present invention leads to prophylactic and therapeutic intervention by induction of autophagy including neurodegenerative disorders, cancers, ageing, obesity, inflammatory bowel disease (IBD), NASH and infections by bacterial, viral and parasitic pathogens and diseases associated with Prion.

In another aspect the invention provides a method of inducing autophagy, comprising administering to a person in need thereof a therapeutically effective amount which can be 1-99% of the peptides claimed in the patent. Applications of the present invention include both prophylactic and therapeutic utilities of the composition of the present invention in need of enhanced autophagy and include diseases and pathologies where the upregulation of autophagy is therapeutically beneficial, including infection with intracellular pathogens, neurodegenerative diseases, cancers, cardiomyopathy, antiviral activity, and aging.

Advantages:

Without being limited to theory, the peptides can be used individually, in-combination or in a composition to induce autophagy. These peptides can also facilitate the study of autophagy and its mechanism in diseased and non-diseased states. The peptides of the present invention can be used for enhancement of autophagy in diseased conditions including neurodegenerative disorders, cancers, ageing, obesity, inflammatory bowel disease (IBD), NASH and infections by bacterial and viral pathogens.

### EXAMPLES

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein, including citations therein, are hereby incorporated by reference in their entirety for all purposes.

### Example 1: Step 1: Molecular Modelling and Protein-Protein Docking to Achieve the most likely Interaction Mode of GAPR-1 and Beclin-1

The coordinates of the proteins GAPR-1 and beclin-1 ECD were obtained from the Protein DataBank (PDB) with PDB ids 4aiw and 4ddp, respectively. Missing residues, side chains and hydrogens are added using Prime module provided in Schrodinger package. The structure will be further minimized using OPLS3.0 force field. Binding modes of the two proteins were predicted using swarmdock and Piper modules implemented in Schrodinger-2016 package.

### Example 2: Step 2: Prediction of Hot-Spot Amino Acids

Long MD simulation of 100 ns was performed with the Amber 16.0 software and Ambertools 16. The tleap program implemented in Amber 16 will be used to assign parameters from FF14SB force field to the molecular systems. The theoretical intermolecular binding free energy between protein and peptide, over the course of simulation time, will be calculated using the MM-PBSA/MM-GBSA approaches implemented in Amber 16. Thermodynamic profiling will be performed for each residue in the protein-peptide complex to identify those amino-acids that are crucial for the binding of peptide with the protein. These residues are called “hot-spot” amino acids.

### Example 3. Step 3: Binding of Peptides to Gapr-1.

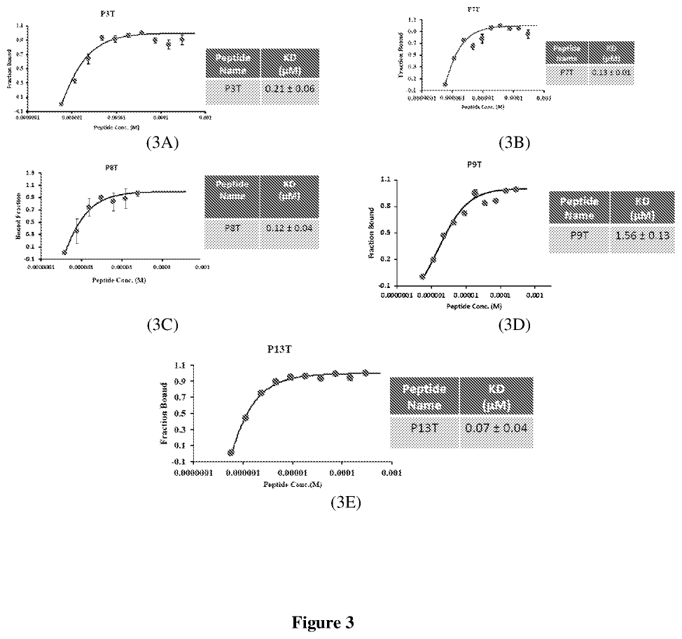

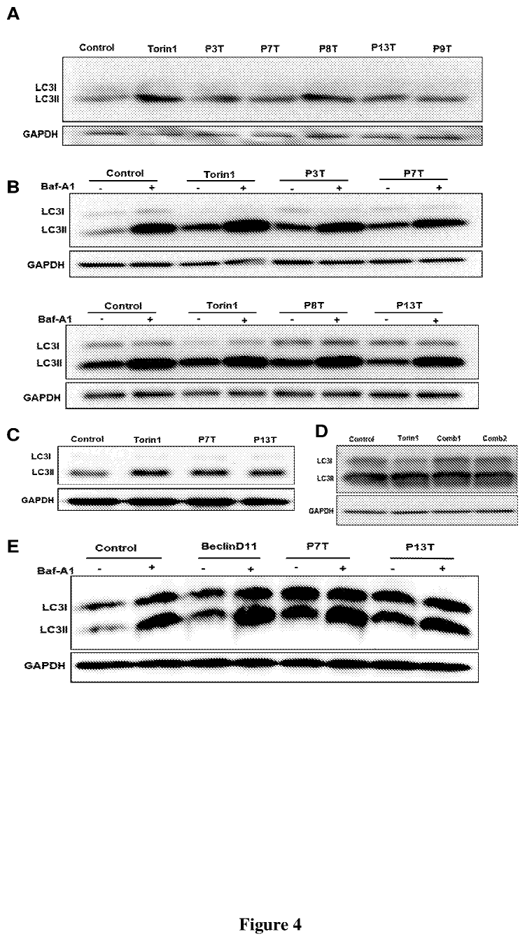

Peptide P3T was found to bind to purified Gapr-1 with the Kd of 0.61 μM as shown in FIG. 3.

### Example 4: Effect of Peptides on Autophagy Induction

To identify the cellular autophagy level in response to peptide treatment we monitored the conversion of LC3-I form to itslipidated membrane-bound form LC3-II, which is an indicator of autophagosome accumulation. Huh7 cells were treated with peptides at 10 μM concentration and with Torin (1 μM, positive control) for 6 hrs. The western blot analysis suggests that the following peptide treatments are leading to the accumulation of autophagosomes in the cell. Peptide P3T was found to bind to purified Gapr-1 with the Kd of 0.61 μM. Autophagy induction by PT series of peptides: Huh7 cells were treated with peptides at 10 μM concentration and torin (1 μM) for 6 hrs and lysed in lysis buffer (1 mM EDTA, 20 mM Tris-HCl pH 7.4, 250 mM NaCl, PMSF, and PI). Western blot was developed by using LC3 and GAPDH antibody. T. Torin, LC3-I and LC3-II, Microtubule-associated protein 1A/1B-light chain 3, GAPDH (internal control) as shown in FIG. 4.

### Example 5: Evaluation of the Peptides of the Present Invention for Induce mTor Independent Autophagy

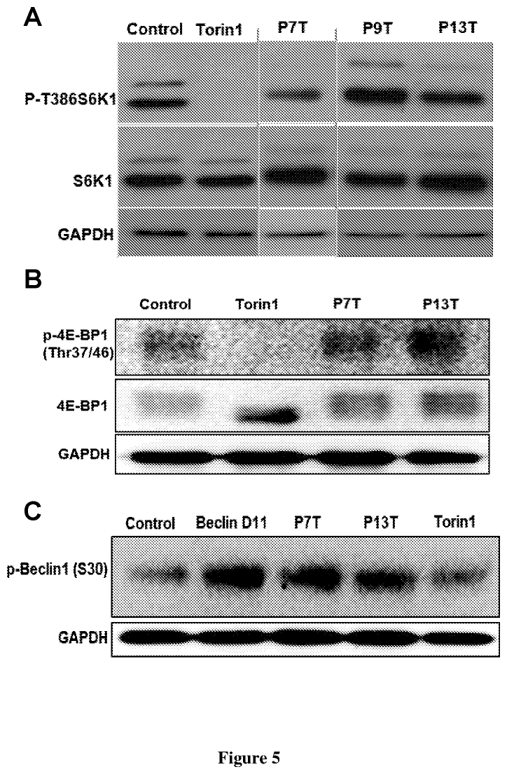

Huh7/HeLa cells were treated with peptides 10 μM for 6 h and protein lysates were analysed by western blotting using pS6Kinase, S6Kinase, p-4E-BP1, 4E-BP1 and GAPDH antibodies. Torin 1 was used as positive control. Peptide treatments did not result in dephosphorylation of S6Kinase and 4E-BP1, indicating that the peptides induce autophagy through mTOR-independent mechanism as shown in FIG. 5 (A-B).

### Example 6: Evaluation of the Therapeutic Effect of Autophagy Inducing Peptides

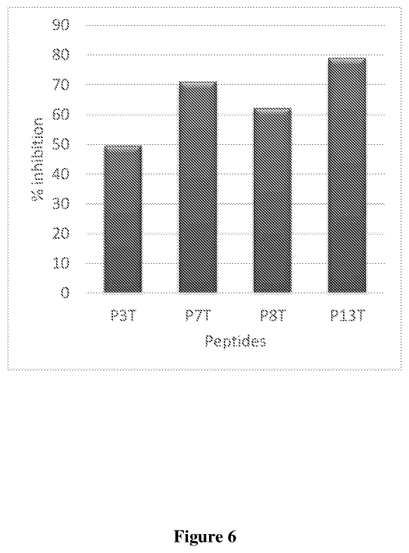

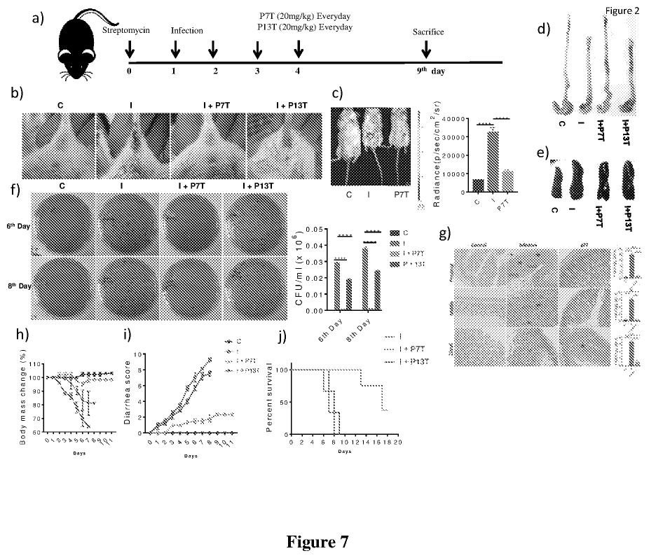

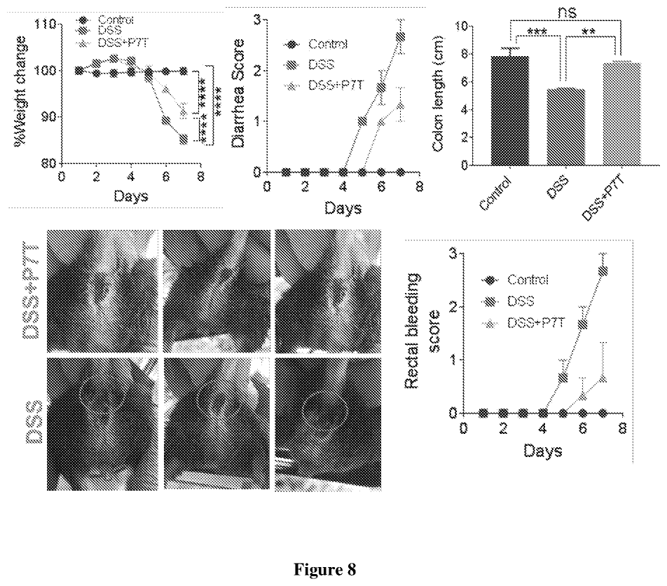

To test the therapeutic potential of autophagy inducing peptides, they were tested in in vitro and in vivo models of typhoid. mice models. For in vitro study, human monocytes derived THP-1 cell line was used. The cell line procured from the ATCC was cultured aseptically in 10% fetal bovine serum (FBS)-containing DMEM cell culture medium at 37° C. in 5% CO2. To differentiate THP-1 into macrophages, non-adherent THP-1 cells were seeded at a density of 5×104 cells per well in flat-bottom 96-well plates and treated with 20 ng/ml PMA for overnight and washed with PBS to remove traces of PMA. Thereafter the cells were washed with phosphate-buffered saline (PBS) and then incubated in RPMI containing 80 μg/ml Gentamicin for 1 h at 37° C. followed by a RPMI wash containing 10 μg/ml Gentamicin. The infected THP-1 cells were then used directly or lysed in 0.1% Triton X-100. From the lysate of the THP-1 cells, colony plating was carried out for Salmonella typhi on SS agar plates. After 24 h, the number of colonies was counted manually as described earlier (Valle E et al., 2005). Peptides P3T, P7T, P8T and P13T were observed to inhibit S. typhimurium by 49.5%, 71%, 62.16% and 79%, respectively. The results are depicted at FIG. 6.

### Example 7: In Vivo Effect of p7T on S. typhimurium infection and Disease Progression in FVB Mice

For the in vivo studies, mouse S. typhimurium (strain Xen33) infection carried out as per the previous protocol of Griffin et al., 2011. In brief, mice were pre-treated with streptomycin. Water and food were withdrawn before 4hours of infection. For the experiments, 6-8 weeks of FVB male mice were utilized in a group of 5. Mice related experiments and its maintenance were carried out in a small animal facility at THSTI, Faridabad. Since P7T and P13T showed more than 70% inhibition, these peptides were selected for in vivo treatment. FVB mice were infected orally with Xen-33(S. typhimurium) and its bioluminescence was measured by in vivo imaging with IVIS Lumina (Perkin Elmer) and quantified with living image software version 4.2. Bioluminescent levels were measured as radiance (p/sec/cm²/sr), by using a pseudo scale for measurement in which blue represents low intensity and red represents high intensity. Posts in vivo imaging, the bacterial burden in faecal sample were also measured. A significant decrease in bacterial burden was observed in mice treated with P7T.

Mice having Salmonella typhimurium infection or non-infected were sacrificed and their intestine was removed for immunohistochemistry studies. Intestines were washed with PBS and fixed in 10% formaldehyde. Thereafter, fine-sectioned on a microtome. Tissue blocks were prepared on Paraffin wax and then hematoxylin and eosin (HE) staining was carried out. The finally stained sections were then observed, and images were acquired by using the Ti Eclipse Nikon microscope. The scoring was done by random scoring by different individuals. It was found that the histopathology score was significantly less in P7T treated mice compare to infection.

In vivo effect of p7T treatment on S. typhimurium infection and disease progression in FVB mice. Recovery of chronically S. typhimurium infected FVB mice was studied in the presence of P7T&P13T. (a panel) Show that pictorial representation of the dose regime for the establishment of infection and P7T and P13T treatment. Panel b, shows mouse images indicating diarrhoea index for 8^{th} day post-infection. The FIG. 7 (Salmonella infection) shows in vivo imaging and intensity quantitation of FVB mice infected with S. typhimurium strain (Xen33) expressing luciferase on 8^{th} day. It also found that the length of the colon was significantly reduced in untreated mice compare to P7T, however difference in splenomegaly was not observed. Colon samples were further examined for histological changes. It is noted that the histopathology score was significantly less in P7T treated mice compare to infection. The stool was collected, plated on SS agar to determine S. typhimurium colony formation. There was significant decrease in bacterial burden in P7T treated mice. Panel h, i & j of FIG. 7 shows the percentage change in body mass, diarrhoea score and percent survival for FVB mice infected with S. typhimurium in the presence of P7T and P13T.

a) Shows the graphic representation of the dose regime for the establishment of infection and treatment. (b) shows mouse images indicating diarrhoea index for 8^{th} post-infection. (c) In vivo imaging and intensity quantitation of mice infected with S. typhimurium strain (Xen33) expressing luciferase on the 8^{th} day (n=5 mice per group). (d-e) The image shows the length of the colon, spleen enlargement isolated from treated or untreated mice. f) The stool was collected and plated on SS agar to determine S. typhimurium colony formation. (g) H & E staining (10×) of the proximal, middle and distal colon regions showed ulcers (arrow), inflammation (line), edema (circle) of different groups along with their histological scores. (i) Immunohistochemistry images (40×) showing expression of HIF-1α and its quantification in the colon (n=5). H, i & j shows the percentage change in body mass, diarrhoea score, and percent survival (Kaplan Meier survival analysis) for FVB mice infected with S. typhimurium (n=5 mice per group). Data representative of triplicates and repeated three times each individual experiments*P<0.05, **P <0.01, ***P<0.001, **** P<0.0001, ns, non-significant. (Student's t-test and one-way ANOVA).

### Example 8: In Vivo Effect of P7T for IBD in Mice Model

DSS (Molecular weight 40,000, Sigma) was dissolved in drinking water to 2.0% (w/v) was given to mice (6-8 weeks) for seven days. The body weight of experimental animals was measured on a daily basis. In the P7T (20mg/kg) treatment group, animals were given intraperitoneal injections on day-1, 2 and 5. Spleen, peripheral lymph nodes (PLNs), mesenteric lymph nodes (MLNs) and colon tissues were harvested for FACs and real time analysis, and colon length measured. Distal segment of colon were used for H&E staining. Stool was collected from DSS, control and treated mice at the time of sacrifice. The stool samples were stored at −80 degree centigrade for 16S marker gene sequencing analysis. Mice were monitored daily or every two days for diarrhea and anal bleeding scores. A number to each observation was given blindly from 1 to 3 based on disease severity. Stool consistency was measured for diarrhea score: score 0, solid pellet; score 1, soft but pellet-shaped; score 2, loose stool with some liquidity; score 3, watery stool. For diarrhea score mice bedding, sticky unformed stool on the cage walls were also taken into consideration. The score for anal bleeding was given as score 0, no visual blood detected in the stool; score 1, light reddish blood streaks in the stool (rub stool on white paper and check for the light stain if not able to see the streaks); score 2, occult blood positive with visual blood in the stool; score 3, bright red color blood in stool (anus completely covered by blood).

### Example 9: Comparison of Illustrative Peptides of the Present Invention with Active Prior Art Peptides

Illustrative peptides of the present invention, namely P7T, P13T, P8_d1 were compared with the prior art peptides Tat D11 and Torin. Tat D11 is a known peptide reported for its autophagy effect.

HeLa cells were treated with peptides and Tat-D11 for 10 μM for 6 h and protein lysates were analysed by western blotting using p-Beclin 1 and GAPDH antibodies. The peptides induced accumulation of lipidated LC3 (LC3-II) and phosphorylation of Beclin1 at levels comparable to Beclin D11, as shown in FIG. 4D and 5C, respectively.

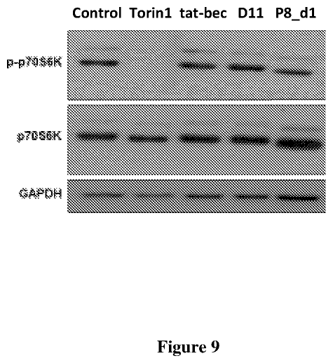

To identify the cellular autophagy levels in response to peptide treatment we monitored the conversion of LC3-I form to its lipidated membrane-bound form LC3-II, which is an indicator of autophagosome accumulation. Huh7/HeLa cells were treated with peptides at 10.0 μM and with Torin 1 (1.0 μM, positive control) for 6 h. Autophagy flux assays were performed by giving Bafilomycin A1 (100 nM) treatment for 2 h before cell lysis (FIG. 9).

From this experiment it can be seen that the peptides of the present invention have enhanced therapeutic efficacy.

### Example 10: Anti-HIV Activity of the Peptides of the Present Invention

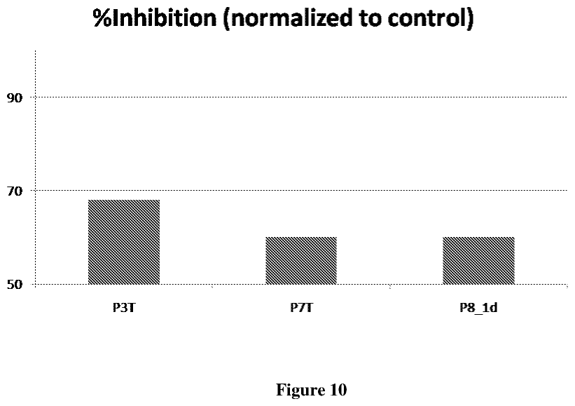

The anti-HIV activity of the peptides of the present invention were tested under virus production and infectivity assay. Single cycle HIV-1 particles were produced from HEK293T by co-transfecting HIV-1 based vectors along with Envelope plasmid in 10 cm plate format. Virus containing cell supernatant were collected 48 hours post transfection and were centrifuged at 300×g for 5 min and filtered using 0.22 um syringe filter. Further virus was quantified using PCR based assay. Following this, viruses were added in TZM-GFP target reporter cells(upon virus entry this cell lines starts expressing GFP) for infection that were seeded 24 hours before infection in a 96-well plate. Prior to virus addition cells were incubated for 4 h with small molecules/peptides (30 uM). Virus infectivity was scored using Thermo Cellinsight cx7 high content imaging platform as the total GFP positive cells. The results are presented at FIG. 10. From the figures, it can be seen that the peptide of the present invention has HIV inhibitory activity.

We claim:

1. Novel engineered peptide for autophagy induction, said peptide being selected from group comprising P3T, P7T, P8T, P9T, P13T and P8_d1 of Seq ID No. 1 to 6 or their combinations.

2. Novel engineered peptide for autophagy induction as claimed in claim 1, wherein the peptide is P7T, of Seq ID No. 2.

3. The peptides as claimed in claim 1, of Seq ID. Nos 1 to 5, wherein, P3T consists of Beclin 1 residues 332-338(PYGNHSY), P7T consists of Beclin 1 residues 282-291(FGTINNFRL), P8T consists of Beclin 1 residues 351-362(LYCSGGLRFFWD), P9T consists of Beclin 1 residues 310-317(QTVLLLHAL) and P13T consists of Beclin 1 residues 329-341(RLVPYGNHSYLES).

4. The peptides as claimed in claim 1 and 3, of Seq ID. Nos 1 to 5, wherein P3T consists of Beclin 1 residues 332-338(PYGNHSY), P7T consists of Beclin 1 residues 282-291(FGTINNFRL), P8T consists of Beclin 1 residues 351-362(LYCSGGLRFFWD), P9T consists of Beclin 1 residues 310-317(QTVLLLHAL) and P13T consists of Beclin 1 residues 329-341(RLVPYGNHSYLES), wherein each terminus flanking the sequence by no more than 12 (or 6, 3, 2, 1 or 0) naturally-flanking Beclin 1 residues, wherein up to six (or 3, 2, 1 or 0) is substituted.

5. The peptides as claimed in claim 1 of Seq ID. Nos 1 to 5, wherein, the peptides are modified to constrain the peptide backbone, selected from the group comprising cyclic peptides/peptidomimetics, inverse peptides, retro-inverse peptides, stapled peptides, which can exhibit enhanced metabolic stability against exopeptidases due to protected C- and N-terminal ends.

6. The peptide as claimed in claims 1, 4 and 5, wherein the peptide is P8_d1, of Seq ID No. 6.

7. The peptide for autophagy induction, as claimed in claim 1, in the form a combination of two or more peptides in any manner selected from group comprising P3T, P7T, P8T, P9T, P13T and P8_d1 of Seq ID No. 1 to 6, preferably the combination is of P7T and P13T or a combination of P3T and P13T.

8. A process for preparing the peptides as claimed in claim 1, by synthetic method or recombinant techniques.

9. A composition comprising any one or a combination of the peptides as claimed in claims 1 to 7 along with pharmaceutically acceptable excipients.

10. The composition as claimed in claim 9, when administered orally, parenterally, or topically.

11. The peptides or combinations thereof as claimed in claim 1 or composition as claimed in claim 9 for induction of autophagy including neurodegenerative disorders, cancers, ageing, obesity, inflammatory bowel disease (IBD), NASH and infections by bacterial, viral and parasitic pathogens and diseases associated with Prion.

12. The peptides or combinations thereof as claimed in claim 1 or composition as claimed in claim 9, for its effect in autophagy.

13. The peptides or combinations thereof as claimed in claim 1 or composition as claimed in claim 9, in an amount ranging from 1 to 100 mg/Kg to a mammal, preferably in an amount ranging from 1 to 50 mg/Kg to a mammal, in an amount ranging from 1 to 25 mg/Kg to a mammal.
